(19) United States
(12) Patent Application Publication
(10) Pub. No.: US 2026/0192053 A1
(43) Pub. Date: Jul. 9, 2026

(54) SAFETY INJECTION DEVICE FOR DELIVERING A DRUG

(71) Applicant: SHL Medical AG, Zug (CH)

(72) Inventor: Daniel Carlsson, Nacka Strand (SE)

(21) Appl. No.: 19/136,387

(22) PCT Filed: Dec. 1, 2023
(86) PCT No.: PCT/EP2023/083885

§ 371 Date: Jun. 6, 2025

(30) Foreign Application Priority Data
Dec. 15, 2022 (EP) 22213896.8

(51) Int. Cl. A61M 5/315; A61M 5/32

(52) U.S. Cl. CPC A61M 5/31511; A61M 2005/3239

(57) ABSTRACT

The present disclosure provides a safety device for delivering a drug. The safety device extends generally along a longitudinal axis and has a proximal end configured to point towards a dose delivery site during use of the safety device, and a distal end pointing away from the dose delivery site. The safety device includes a drug container holder, generally cylindrical and extending along the longitudinal axis, and configured to receive and hold a drug container. The safety device further includes a ring-shaped rotator arranged around a portion of the drug container holder. The safety device further includes a manually activated plunger assembly, extending along the longitudinal axis, the plunger assembly comprising a plunger rod capable of acting on a plunger inside a barrel of the drug container for expelling a dose of the drug, and a plunger head formed on a distal end of the plunger rod and configured to be manually pushed in a proximal direction to expel the drug. The plunger head includes at least a fin extending towards the proximal end. The rotator includes an outer pocket designed to receive the fin, and the shapes of the fin and of the outer pocket are such that a movement of the plunger head towards the rotator along the longitudinal axis triggers a rotation of the rotator when the fin contacts a corner of the outer pocket.

### TECHNICAL FIELD

The invention relates to safety devices for delivering drugs.

### BACKGROUND

Injection devices typically fall into two categories—manual devices and autoinjectors. A conventional autoinjector may provide the force for administering the injection by a spring, and a trigger button or other mechanism may be used to activate the injection. On the contrary, in a conventional manual device, a user must provide force to drive a drug through a needle. This is typically done by some form of button/plunger rod that has to be continuously pressed during the injection.

Safety devices are manual injection devices providing needle safety. They are adapted to avoid accidental needle injuries before, during and after an injection of a medication or drug contained in a pre-filled drug container. Safety devices covering a needle of a pre-filled drug container before and after use are well known. When the needle is covered, the device is said “locked-out”.

Typically, these devices comprise a needle shield used to surround the needle after injection, the needle shield being either manually moved or moved by the action of a biasing member, for instance a pre-compressed spring. Different type of safety devices known in the state of the provide needle safety by arranging the needle movable relative to a housing, wherein the needle is retracted into the housing after the injection.

It is an object of the present invention to provide a safety device adapted to retract the needle into the housing after the injection.

### SUMMARY

Reference should now be made to the appended claims.

The invention relates to a safety device for delivering a drug, the safety device extending generally along a longitudinal axis and having a proximal end configured to point towards a dose delivery site during use of the safety device, and a distal end pointing away from the dose delivery site, the safety device comprising:
a drug container holder, generally cylindrical and extending along the longitudinal axis, and configured to receive and hold a drug container,
a ring-shaped rotator arranged around a portion of the drug container holder,
a manually activatable plunger assembly, extending along the longitudinal axis, the plunger assembly comprising a plunger rod capable of acting on a plunger inside a barrel of the drug container for expelling a dose of the drug, and a plunger head formed on a distal end of the plunger rod and configured to be manually pushed in a proximal direction to expel the drug,
the plunger head having at least a fin extending towards the proximal end, the rotator comprising an outer pocket designed to receive the fin, and the shapes of the fin and of the outer pocket are such that a movement of the plunger head towards the rotator along the longitudinal axis triggers a rotation of the rotator when the fin contacts a corner of the outer pocket.

In a non-limiting embodiment, a proximally facing edge of the fin is angled relative to planes that are orthogonal to the longitudinal axis.

In a non-limiting embodiment, the plunger head defines an annular recess about the plunger rod and comprises a distal portion extending orthogonal to the longitudinal axis, and the fin extends in the annular recess, parallel to the longitudinal axis, from the distal portion.

In a non-limiting embodiment, the rotator has an outer surface comprising a recess forming the outer pocket, the outer pocket extending from a distal end of the rotator.

In a non-limiting embodiment, an outer surface of the rotator comprises at least a locking knob, and a housing of the safety device has a collar, the collar comprising at least an elongated through aperture forming a track for the locking knob, allowing a rotation of the rotator around the longitudinal axis but preventing an axial displacement of the rotator with regards to the housing.

In a non-limiting embodiment, a housing of the safety device comprises at least a handle, and the collar is substantially annular and extends from the handle towards the distal end, around the longitudinal axis.

In a non-limiting embodiment, an outer surface of the drug container holder has a distal elongated ledge extending parallel to the longitudinal axis, and the rotator comprises a receiving element designed to receive a distal extremity of the distal elongated part in a non-rotated position of the rotator, and a first space forming a track for the distal elongated part in a rotated position of the rotator.

In a non-limiting embodiment, an inner surface of the rotator comprises at least a recess forming an inner pocket, the receiving element protruding from said inner pocket, and the first space is defined between a first lateral edge of the inner pocket and the receiving element, the first space extending fully across the rotator along the longitudinal axis.

In a non-limiting embodiment, the drug container holder comprises two flexible lock-out fingers joining each other at a connection area corresponding to a proximal extremity of the distal elongated part, the lock-out fingers being adapted to cross the first space when the drug container holder is moved relative to the rotator only in a distal direction.

In a non-limiting embodiment, the outer surface of the drug container holder comprises a proximal elongated ledge extending parallel to the longitudinal axis, the first space forming a track for the proximal elongated part in the rotated position of the rotator after the flexible lock-out fingers have passed the rotator.

In a non-limiting embodiment, the proximal elongated part comprises a protrusion configured to prevent an axial displacement of the drug container holder in the proximal displacement once the flexible lock-out fingers have passed the rotator.

In a non-limiting embodiment, the drug container holder comprises at least two flexible locking arms extending parallel to the longitudinal axis from a distal end of the drug container holder, the locking arms being designed to surround a portion of the plunger rod, the rotator being designed to surround the locking arms and prevent the locking arms from flexing radially outward.

In a non-limiting embodiment, the locking arms have an elongated inner recess designed to complement strips extending along the plunger rod.

In a non-limiting embodiment, the safety device comprises a flaring neck at the proximal end, the flaring neck defining a proximally facing injection site support area, and a distally facing hand resting area.

In the present disclosure, when the term “distal direction” is used, this refers to the direction pointing away from the dose delivery site during use of the safety device. When the term “distal part/end” is used, this refers to the part/end of the safety device, or the parts/ends of the members thereof, which during use of the safety device is/are located furthest away from the dose delivery site. Correspondingly, when the term “proximal direction” is used, this refers to the direction pointing towards the dose delivery site during use of the safety device. When the term “proximal part/end” is used, this refers to the part/end of the safety device, or the parts/ends of the members thereof, which during use of the safety device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Finally, when a component is said to move proximally, distally, axially in a proximal direction, or axially in a distal direction, the movement is relative to the housing of the injection device, unless mentioned otherwise.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1 shows an injection device comprising a safety device according to a non-limiting embodiment of the invention, in a packaged state.

FIG. 2 is an exploded view of the injection device of FIG. 1.

FIG. 3 is a cross-sectional view of the injection device of FIG. 1, after a cap member of the safety device has been removed but before activation.

FIG. 4 shows a drug container holder of the safety device of FIG. 1.

FIG. 5 shows a plunger assembly of the safety device of FIG. 1.

FIG. 6 is a cross-sectional view of the injection device of FIG. 1, after drug has been expelled but before the device lock-out.

FIG. 7 is a close-up view of a proximal part of FIG. 3.

FIGS. 8A and 8B show a rotator of the safety device of FIG. 1.

FIG. 9 shows a housing of the safety device of FIG. 1.

FIGS. 10A and 10B show a distal part of the injection device of FIG. 1, when the drug expelling is almost over and when the drug expelling is over.

FIGS. 11A, 11B, 11C and 11D show the injection device of FIG. 1, at different stages.

FIGS. 12A and 12B show an injection device comprising a safety device according to an alternative non-limiting embodiment of the invention.

FIGS. 13A, 13B and 13C show three steps of an injection sequence intended to be performed with the injection device of FIGS. 12A and 12B.

### DETAILED DESCRIPTION

To provide an overall understanding of the safety device described herein, certain illustrative embodiments will now be described.

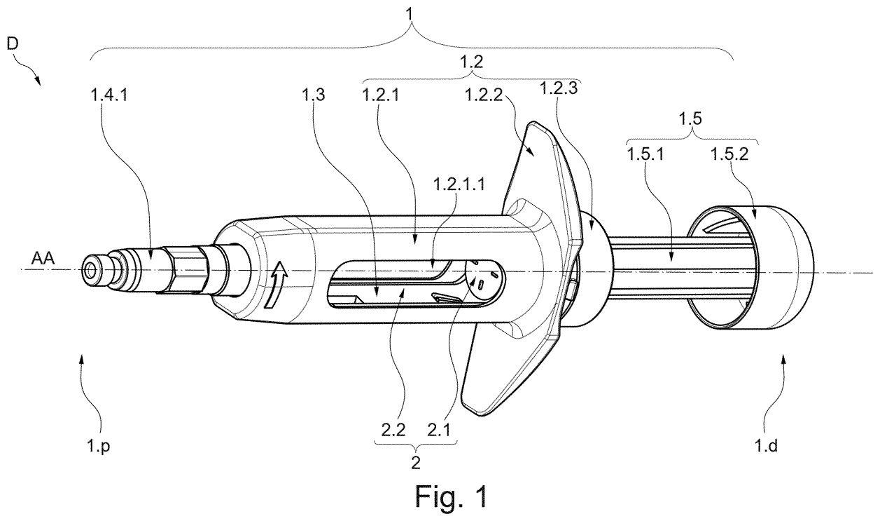

FIG. 1 shows an injection device D in a packaged state as it would be presented to a user prior injection.

The injection device D comprises a safety device 1 and a drug container 2, for instance a cartridge. The drug container 2 contains a set dosage of a drug.

The safety device 1 extends along a longitudinal axis AA between a proximal end 1.p, where the drug is ejected, and a distal end 1.d. The proximal end 1.p is configured to point towards a dose delivery site (i.e., a patient's skin part) during use of the safety device 1, and a distal end 1.d pointing away from the dose delivery site.

The safety device 1 comprises a housing 1.2. The housing 1.2 comprises a case 1.2.1. The case 1.2.1 is generally tubular, but other shapes are possible. The case 1.2.1 extends along the longitudinal axis AA. The case 1.2.1 is hollow and defines an outer chamber. The outer chamber is dimensioned to receive a drug container holder 1.3, itself dimensioned to receive and hold the drug container 2. The case 1.2.1 comprises an optional window 1.2.1.1 through which a user can see the drug to be injected. The housing 1.2 further comprises two diametrically opposite handles 1.2.2, extending from the case 1.2.1 orthogonally to the longitudinal axis AA. Another number of handles or no handles at all is possible. Finally, the housing 1.2 comprises a collar 1.2.3, substantially annular, extending from the handles 1.2.2 towards the distal end 1.d and around the longitudinal axis AA.

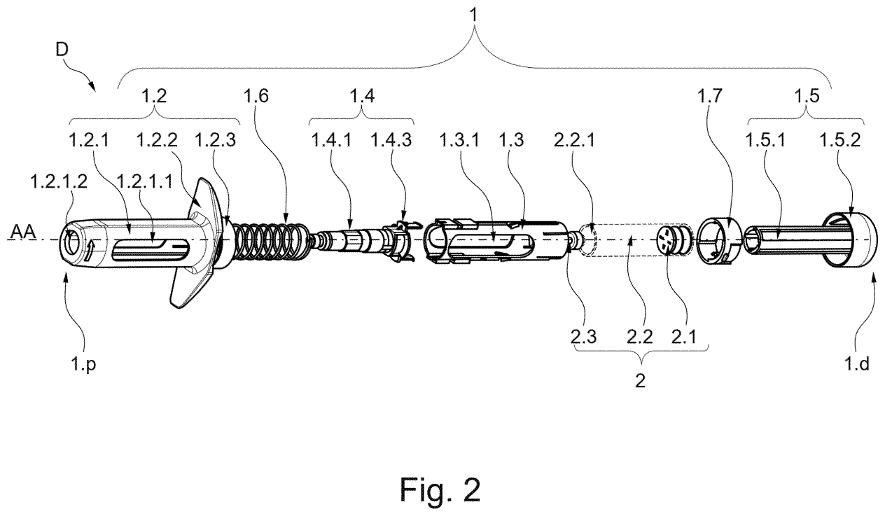

The safety device 1 comprises the drug container holder 1.3, configured to hold the drug container 2 within the housing 1.2, depicted in more details in FIG. 2.

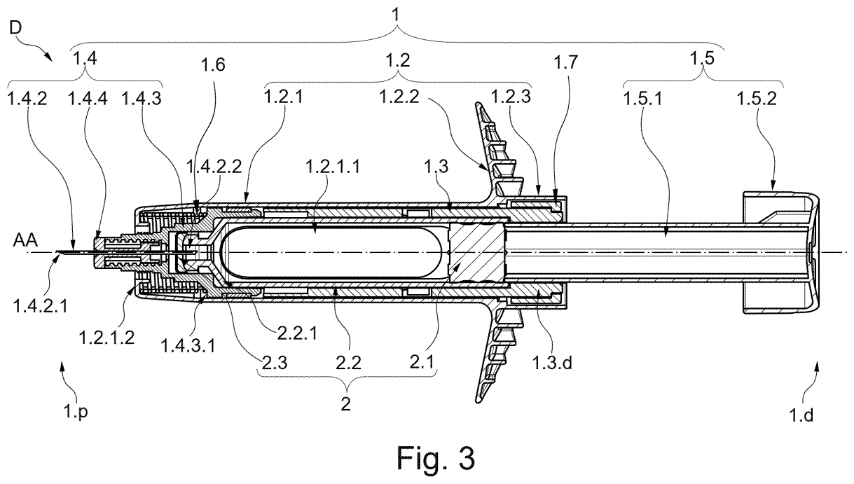

The safety device 1 comprises a cap member 1.4.1 that is part of a needle assembly 1.4 shown in more details with reference to FIG. 2. The cap member 1.4.1 is configured to cover a needle 1.4.2 visible in FIG. 3. The cap member 1.4.1 is removably attached to the rest of the needle assembly 1.4. When the cap member 1.4.1 is removed from the rest of the needle assembly 1.4, the needle 1.4.2 is exposed, as visible in FIG. 3.

The safety device 1 includes a plunger assembly 1.5, including a plunger rod 1.5.1 and a plunger head 1.5.2 (also known as thumb rest). The plunger head 1.5.2 is formed on a distal end of the plunger rod 1.5.1. The plunger rod 1.5.1 is capable, during a displacement from a starting position to an ending position along the longitudinal axis AA, of pushing a plunger 2.1 of the drug container 2 inside a barrel 2.2 of the drug container 2, towards the proximal end 1.p, for expelling a dose of drug through the needle 1.4.2.

To deliver an injection using the injection device D, a user can grab the handles 1.2.2 of the housing 1.2, remove the cap member 1.4.1 from the needle assembly 1.4, insert the needle 1.4.2 into a delivery site, and deliver the injection by pressing the plunger head 1.5.2 of the plunger assembly 1.5 towards the housing 1.2, that is to say in the direction of the proximal end 1.p.

FIG. 2 is an exploded view of the injection device D, showing elements that are not visible in FIG. 1.

The drug container 2 comprises the aforementioned barrel 2.2, generally cylindrical although other shapes are possible, the aforementioned plunger 2.1, located inside the barrel 2.2 and configured to be axially pushed by the plunger rod 1.5.1 until it reaches a neck 2.2.1 at a proximal end of the barrel 2.2, and a membrane 2.3 closing the neck 2.2.1.

The drug container holder 1.3 is generally tubular, although other shapes are possible. The drug container holder 1.3 is hollow and defines an inner chamber. The inner chamber is dimensioned to receive the drug container 2. The drug container holder 1.3 comprises an optional window 1.3.1, configured to be aligned with the optional window 1.2.1.1 of the case 1.2.1 of the housing 1.2, through which a user can see the drug to be injected.

The safety device 1 further comprises the needle assembly 1.4. The needle assembly 1.4 comprises a retainer member 1.4.3 configured to be connected to the drug container holder 1.3, as visible in FIG. 3. Moreover, the case 1.2.1 of the housing 1.2 comprises a proximal opening 1.2.1.2 through which the needle assembly 1.4 extends.

The safety device 1 further comprises a biasing member 1.6, e.g., which is a coil spring in the described non-limiting embodiment.

Finally, the safety device 1 comprises a rotator 1.7, substantially annular and extending around the longitudinal axis AA.

FIG. 3 is a cross-sectional view of the injection device D showing the plunger rod 1.5.1 in the starting position, i.e., before activation of the injection device D, with the cap member 1.4.1 removed from the needle assembly 1.4.

The needle assembly 1.4 comprises a hub 1.4.4 coaxially located within the retainer member 1.4.3 and holding the needle 1.4.2. The cap member 1.4.1 is configured to be interactively connected to both the hub 1.4.4 and to the retainer member 1.4.3. The needle has a proximal pointed end 1.4.2.1 and distal pointed end 1.4.2.2. The distal pointed end 1.4.2.2 of the needle 1.4.2 is configured to penetrate the membrane 2.3 arranged at the proximal end of the barrel 2.2 when the cap member 1.4.1 is removed.

The biasing member 1.6 is in a compressed or loaded state. The biasing member 1.6 is positioned between a distally facing surface of a proximal wall of the case 1.2.1 of the housing 1.2, the wall defining the previously mentioned opening 1.2.1.2, and a proximally facing surface 1.4.3.1 of the retainer member 1.4.3.

The rotator 1.7 is positioned within the collar 1.2.3 of the housing 1.2, and around a distal end 1.3.d of the drug container holder 1.3.

The plunger 2.1 of the drug container 2 is located at a distal end of the barrel 2.2.

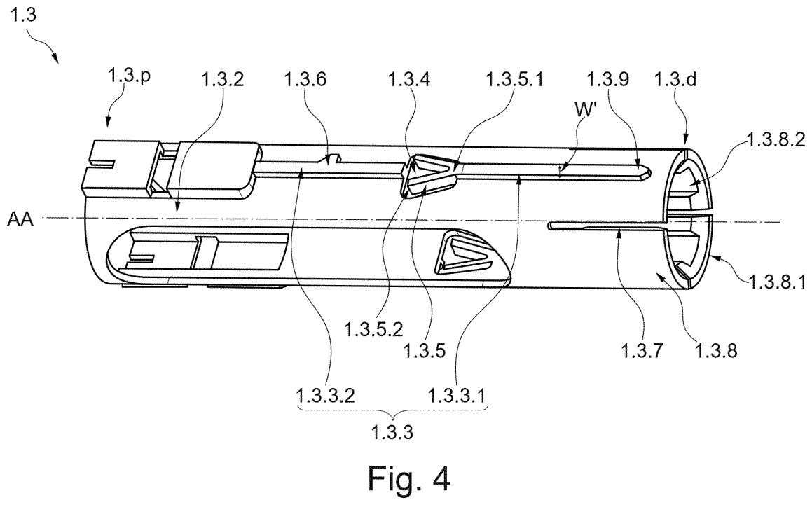

FIG. 4 shows the drug container holder 1.3 according to the invention.

The drug container holder 1.3 is substantially cylindrical and extends between a proximal end 1.3.p and a distal end 1.3.d, along the longitudinal axis AA. The outer surface 1.3.2 of the drug container holder 1.3 has two diametrically opposite and identical elongated ledges 1.3.3, although only one or more than two ledges is possible. The elongated ledges 1.3.3 extend parallel to the longitudinal axis AA. Each elongated ledge 1.3.3 is discontinuous: it is made of two parts, a distal elongated part 1.3.3.1 and a proximal elongated part 1.3.3.2.

Between the distal elongated part 1.3.3.1 and the proximal elongated part 1.3.3.2, the outer surface 1.3.2 of the drug container holder 1.3 has a through opening 1.3.4. Two lock-out fingers 1.3.5 are located inside said opening 1.3.4. The lock-out fingers 1.3.5 join each other at a connection area 1.3.5.1. Said connection area 1.3.5.1 corresponds to the proximal extremity of the distal elongated part 1.3.3.1. In other words, the distal elongated part 1.3.3.1 is extended by the lock-out fingers 1.3.5. The lock-out fingers 1.3.5 extend in the opening 1.3.4 along the outer surface 1.3.2 of the drug container holder 1.3 from the connection area 1.3.5.1 in the direction of the proximal end 1.3.p of the drug container holder 1.3. Without any mechanical force applied to them, the lock-out fingers 1.3.5 are angled relative to each other. Moreover, the lock-out fingers 1.3.5 are flexible, meaning that an applied mechanical force can decrease their angle, i.e., bring their proximal extremities 1.3.5.2 closer to each other.

The proximal elongated part 1.3.3.2 comprises a protrusion 1.3.6. The distance between the proximal extremities 1.3.5.2 of the lock-out fingers 1.3.5 and the protrusion 1.3.6 is similar to the width of the rotator 1.7, so that, in a final position, the rotator 1.7 can be axially blocked between the proximal extremities 1.3.5.2 of the lock-out fingers 1.3.5 and the protrusion 1.3.6.

Finally, the distal elongated part 1.3.3.1 comprises a distal extremity 1.3.9 that has a semi-circular shape, although other shapes are possible.

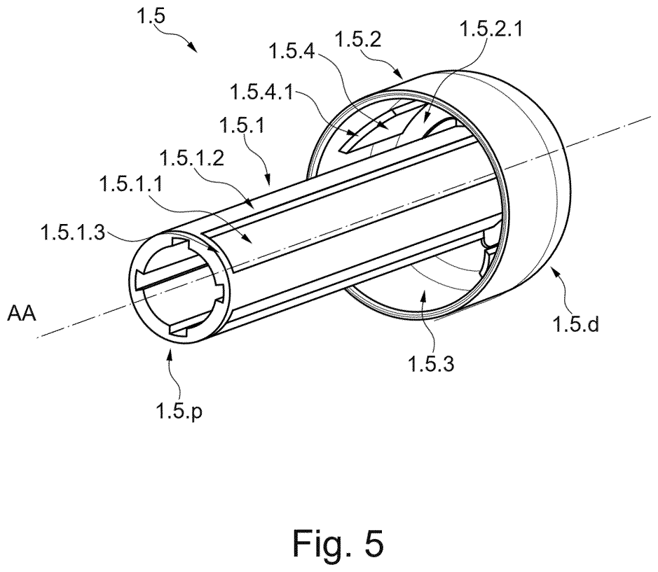

The drug container holder 1.3 further comprises four elongated through openings 1.3.7 starting from the distal end 1.3.d and extending parallel to the longitudinal axis AA, equally spaced from each other. The elongated through openings 1.3.7 define four flexible locking arms 1.3.8, meaning that an applied mechanical force can move the distal extremities 1.3.8.1 of the locking arms 1.3.8 away from each other. Another number of through openings 1.3.7 and locking arms 1.3.8 is possible. Each locking arm 1.3.8 comprises an elongated inner recess 1.3.8.2. The inner recesses 1.3.8.2 start from the distal end 1.3.d and extend parallel to the longitudinal axis AA. The inner recesses 1.3.8.2 are configured to complement the shape of the outer surface of the plunger rod 1.5.1, as explained in reference to FIG. 5.

FIG. 5 shows the plunger assembly 1.5 according to the invention. The plunger rod 1.5.1 has a proximal end 1.5.p and a distal end 1.5.d. The plunger rod 1.5.1 comprises four elongated outer recess 1.5.1.1, extending parallel to the longitudinal axis AA, equally spaced from each other. The outer recesses 1.5.1.1 do not extend fully until the proximal end 1.5.p of the plunger rod 1.5.1, forming a discontinuous holding ring 1.5.1.3 between the proximal end 1.5.p of the plunger rod 1.5.1 and the proximal extremity of the outer recesses 1.5.1.1. The outer recesses 1.5.1.1 define elongated strips 1.5.1.2 all around the plunger rod 1.5.1, each strip 1.5.1.2 being located between two outer recesses 1.5.1.1. The strips 1.5.1.2 on the plunger rod 1.5.1 are dimensioned to be received in the inner recesses 1.3.8.2 of the locking arms 1.3.8 of the drug container holder 1.3.

The assembly process of the proximal end 1.5.p of the plunger rod 1.5.1 to the distal end 1.3.d of the drug container holder 1.3 comprises:
Flexing the locking arms 1.3.8 radially outward,
Inserting the strips 1.5.1.2 between the outer recesses 1.5.1.1 of the plunger rod 1.5.1 in the inner recesses 1.3.8.2 of the locking arms 1.3.8 of the drug container holder 1.3,
Setting the rotator 1.7 around the locking arms 1.3.8, as visible in FIG. 3.

When the rotator 1.7 is assembled around the locking arms 1.3.8 of the drug container holder 1.3, the locking arms 1.3.8 cannot radially flex. Thus, because of the holding ring 1.5.1.3 at the proximal end 1-5.p of the plunger rod 1.5.1, the plunger rod 1.5.1 and the drug container holder 1.3 cannot move in the distal direction relative to the rotator 1.7.

The plunger head 1.5.2 is formed on the distal end 1.5.d of the plunger rod 1.5.1. The plunger head 1.5.2 is substantially cup-shaped and defines an annular recess 1.5.3 about the plunger rod 1.5.1.

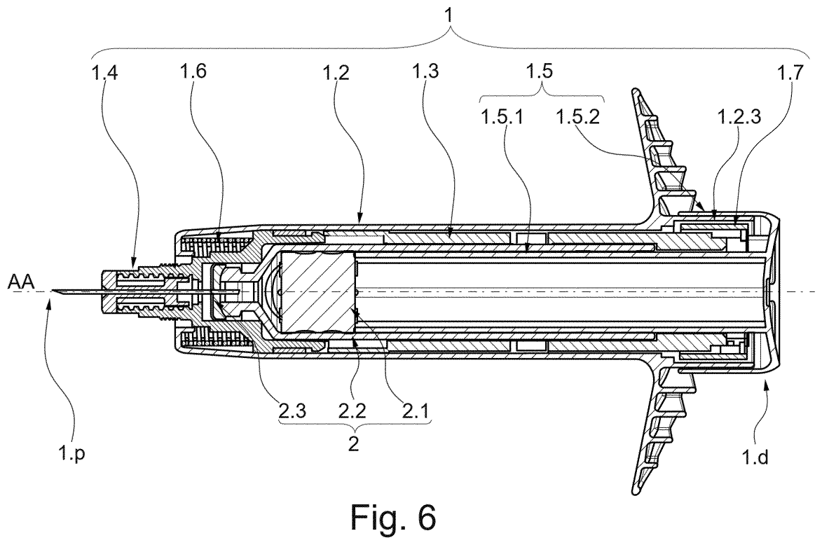

FIG. 6 is a cross-sectional view of the injection device D showing the plunger rod 1.5.1 in the ending position, with the biasing member 1.6 still in a compressed state. Compared to the position depicted in FIG. 3, the plunger 2.1 of the drug container 2 is now located at the proximal end of the barrel 2.2, meaning that drug has been fully expelled from the barrel 2.2.

As shown in FIG. 6, the annular recess 1.5.3 of the plunger assembly 1.5 is dimensioned to receive the collar 1.2.3 of the housing 1.2, the rotator 1.7 and the distal end 1.3.d of the drug container holder 1.3, when the plunger rod 1.5.1 nears and reaches the ending position, i.e., when the plunger rod 1.5.1 is advanced from its starting position to expel drug from the injection device D.

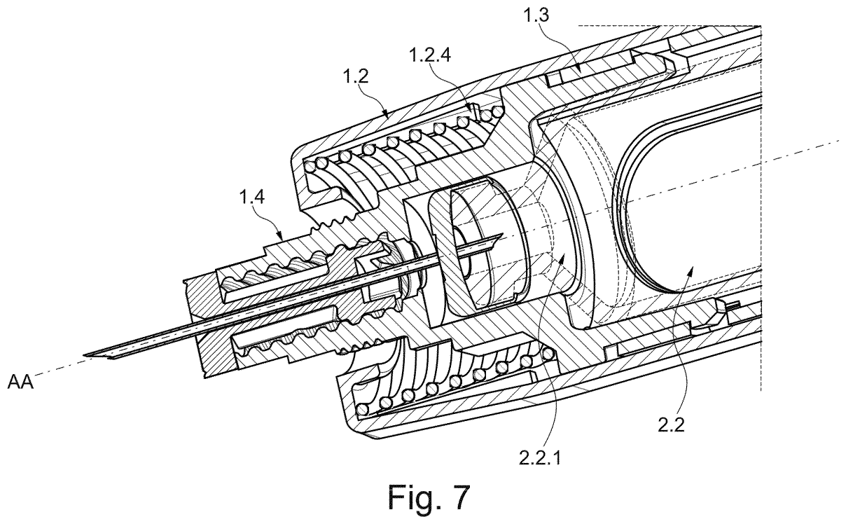

It can be noted that compared to the position depicted in FIG. 3, the needle assembly 1.4, the drug container 2 and the drug container holder 1.3 have moved axially in the proximal direction. The reason will be explained with reference to FIG. 7.

FIG. 7 is a cross-sectional view of a proximal part of the injection device D, in which the plunger rod 1.5.1 is in the starting position, with the cap member 1.4.1 removed from the needle assembly 1.4.

From the starting position of the plunger rod 1.5.1, when a user pushes axially the plunger assembly 1.5 in the proximal direction, the plunger 2.1 in the barrel 2.2 of the drug container 2 is subsequently pushed axially in the proximal direction. Since the biasing member 1.6 has a lower resistance than the force required to expel the drug, the drug container 2 and the needle assembly 1.4 move axially in the proximal direction. Since the drug container holder 1.3 is connected to the needle assembly 1.4, the drug container holder 1.3 also moves axially in the proximal direction. The proximal movement of the needle assembly 1.4, the drug container 2 and the drug container holder 1.3 is stopped when the retainer member 1.4.3 hits a stop element 1.2.4 in the housing 1.2, as depicted in FIG. 7. This behaviour ensures that the needle extension, that is to say the distance from the proximal opening 1.2.1.2 or the housing 1.2 to the proximal end of the needle 1.4.2.1 is set correctly.

Coming back to FIG. 5, the plunger head 1.5.2 comprises a distal portion 1.5.2.1, substantially in shape of a disk although other shapes are possible, which extends orthogonal to the longitudinal axis AA. The plunger head 1.5.2 comprises two fins 1.5.4 extending in the annular recess 1.5.3, parallel to the longitudinal axis AA, from the distal portion 1.5.2.1 of the plunger head 1.5.2.

Another number of fins is possible. Each fin 1.5.4 has a proximally facing edge 1.5.4.1 that is angled relative to planes that are orthogonal to the longitudinal axis AA, meaning the edge is not comprised in any plane orthogonal to the longitudinal axis AA. A plane orthogonal to the longitudinal axis AA is called transversal plane.

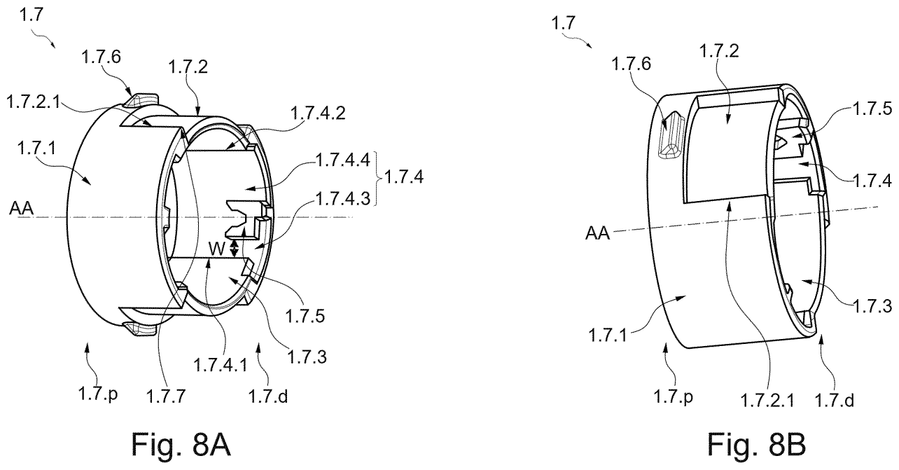

FIGS. 8A and 8B show the rotator 1.7 according to the invention.

The rotator 1.7 has a proximal end 1.7.p and a distal end 1.7.d. The rotator 1.7 is substantially annular and extends around the longitudinal axis AA. The rotator 1.7 has an outer surface 1.7.1 comprising two recesses forming two outer pockets 1.7.2. The outer pockets 1.7.2 extend until the distal end 1.7.d of the rotator 1.7, so that each outer pocket 1.7.2 can receive a fin 1.5.4 of the plunger head 1.5.2 when the plunger rod 1.5.1 nears and reaches the ending position. Each outer pocket 1.7.2 further comprises two lateral edges 1.7.2.1 extending substantially parallel to each other.

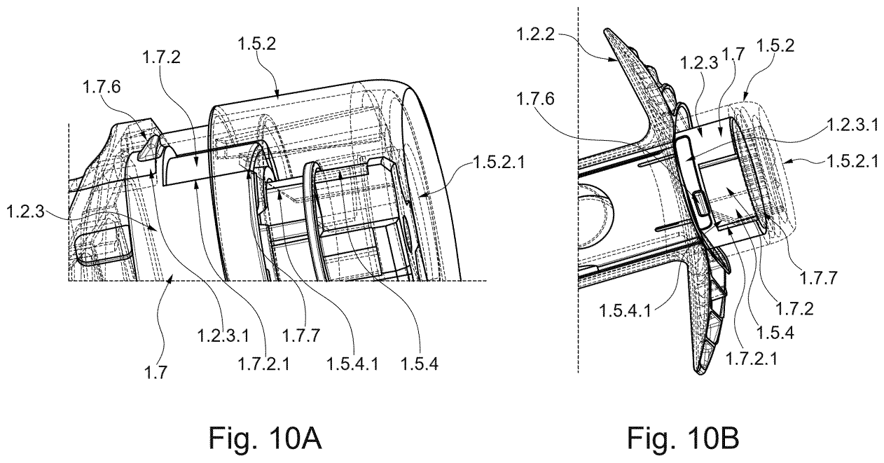

Besides that, the outer pockets 1.7.2 and the fins 1.5.4 are, in the starting position of the plunger rod 1.5.1, angled relative to each other in a way that when a fin 1.5.4 reaches its associated outer pocket 1.7.2 through an axial movement in the proximal direction of the plunger assembly 1.5, the edge 1.5.4.1 of the fin 1.5.4 contacts a corner 1.7.7 between one of the lateral edges 1.7.2.1 of the outer pocket 1.7.2 and the distal end 1.7.d of the rotator 1.7, as shown in FIG. 10A. Thus, when the axial movement of the plunger rod 1.5.1 continues, the rotator 1.7 is forced to rotate around the longitudinal axis AA, as shown in FIG. 10B.

The rotator 1.7 further comprises an inner surface 1.7.3 comprising two recesses forming two inner pockets 1.7.4. The inner pockets 1.7.4 extend from the proximal end 1.7.p to the distal end 1.7.d of the rotator 1.7. Each inner pocket 1.7.4 further comprises two lateral edges 1.7.4.1, 1.7.4.2 extending substantially parallel to each other.

The rotator 1.7 further comprises a receiving element 1.7.5 protruding from each inner pocket 1.7.4. Each receiving element 1.7.5 has a shape that complements the shape of the distal extremity 1.3.9 of the distal elongated part 1.3.3.1 of the drug container holder 1.3. In the starting position of the plunger rod 1.5.1, the distal extremity 1.3.9 of the distal elongated part 1.3.3.1 is locked inside the receiving element 1.7.5, which prevents the rotator 1.7 from rotating around the longitudinal axis AA. The rotator 1.7 only becomes free to rotate when the drug container holder 1.3 has moved axially in the proximal direction, as explained previously, the axial displacement being enough to withdraw the distal extremity 1.3.9 of the distal elongated part 1.3.3.1 from the receiving element 1.7.5.

The receiving element 1.7.5 being located in the inner pocket 1.7.4, two spaces are defined in the inner pocket 1.7.4. A first space 1.7.4.3 extends between a first lateral edge 1.7.4.1 and the receiving element 1.7.5, and a second space 1.7.4.4 extends between a second lateral edge 1.7.4.2 and the receiving element 1.7.5. It should be noted that the width W of first space 1.7.4.3 is similar to the width W′ of the distal elongated part 1.3.3.1, shown in FIG. 4.

The rotator 1.7 further comprises two locking knobs 1.7.6 protruding from the outer surface 1.7.1. Another number of locking knobs is possible.

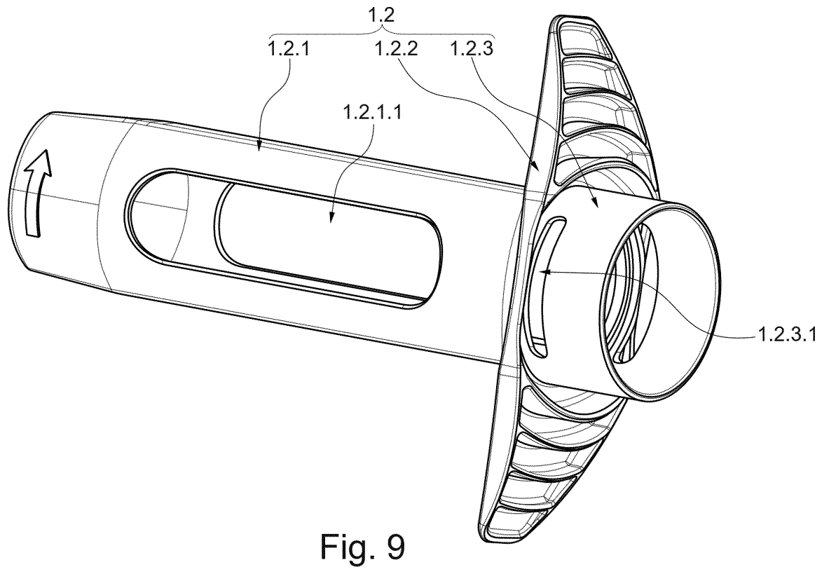

FIG. 9 shows the housing 1.2 according to the invention.

The collar 1.2.3 of the housing 1.2 comprises two elongated through apertures 1.2.3.1, each aperture 1.2.3.1 extending along a portion of a circumference of the collar 1.2.3. Each aperture 1.2.3.1 is dimensioned to receive a locking knob 1.7.6 of the rotator 1.7. The aperture 1.2.3.1 being elongated, a rotation of the rotator 1.7 around the longitudinal axis AA is possible: each aperture 1.2.3.1 forms a track for a locking knob 1.7.6. However, an axial displacement of the rotator 1.7 with regards to the housing 1.2 is impossible.

FIGS. 10A and 10B show a distal part of the injection device D, where the housing 1.2 and the plunger head 1.5.2 are shown as transparent, for clarity purposes.

FIG. 10A represents the plunger head 1.5.2 in a position where the edge 1.5.4.1 of the fin 1.5.4 contacts a corner 1.7.7 between one of the lateral edges 1.7.2.1 of the outer pocket 1.7.2 and the distal end 1.7.d of the rotator 1.7. At that time, the drug container holder 1.3 has already moved axially in the proximal direction, which has removed the distal extremity 1.3.9 of the distal elongated part 1.3.3.1 of the elongated ledge 1.3.3 of the drug container holder 1.3 from the receiving element 1.7.5 of the rotator 1.7. The knob 1.7.6 is located on one side of the aperture 1.2.3.1 on the collar 1.2.3 of the housing 1.2.

FIG. 10B represents the plunger head 1.5.2 in a position where its fin 1.5.4 has now been received in the outer pocket 1.7.2 and the rotator 1.7 has rotated. The knob 1.7.6 is now located on the other side of the aperture 1.2.3.1.

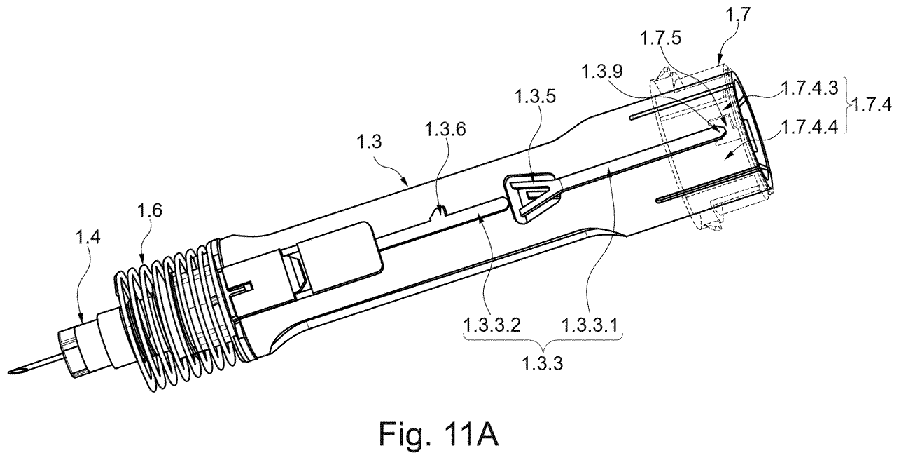

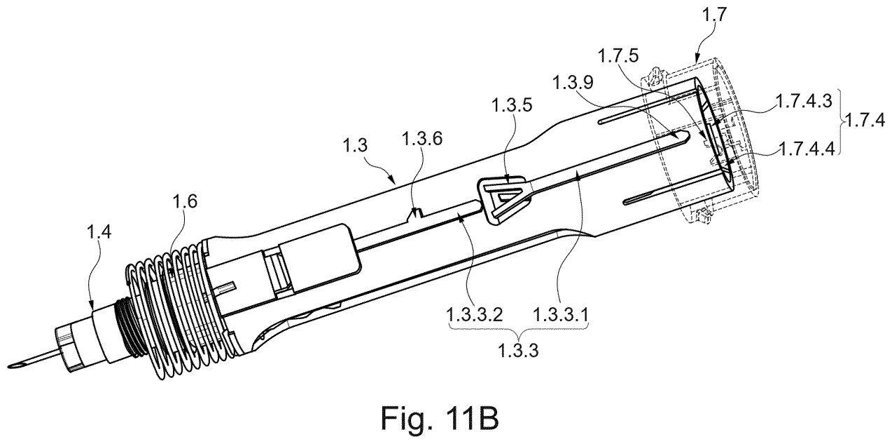

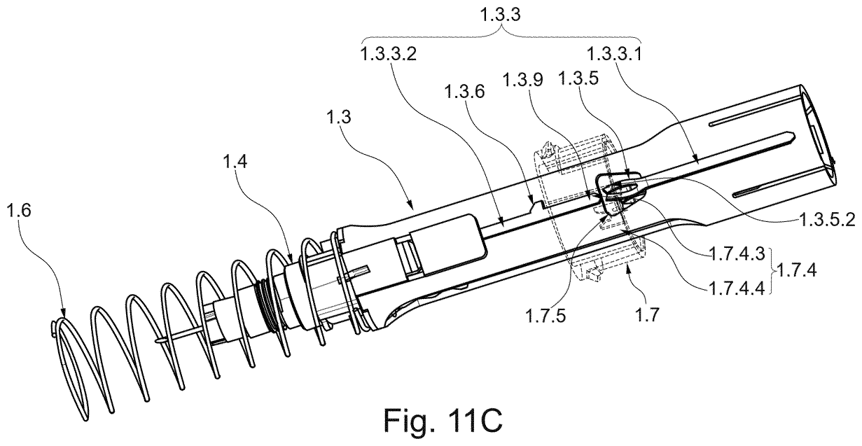

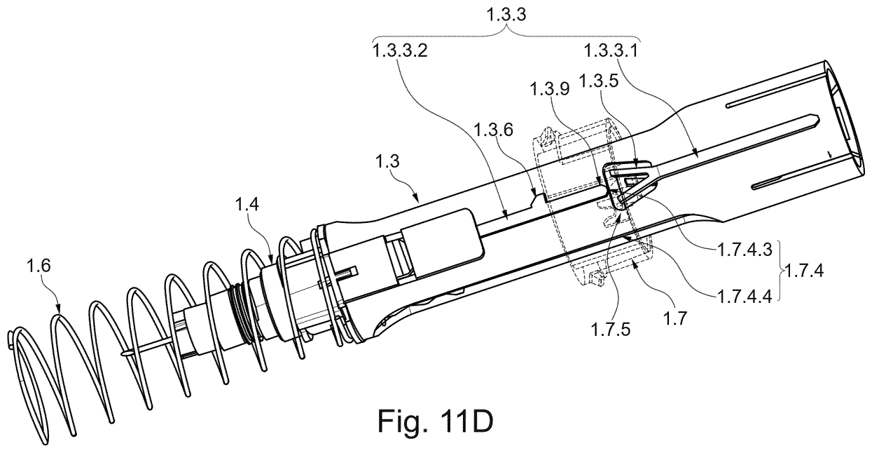

FIGS. 11A, 11B, 11C and 11D show the injection device D at different stages, with the plunger rod 1.5.1 in the starting position, and with the cap member 1.4.1 removed from the needle assembly 1.4. For clarity purposes, the housing 1.2 and the plunger assembly 1.5 are not shown and the rotator 1.7 is shown as transparent.

In FIG. 11A, the injection device D is shown before activation. The plunger rod 1.5.1 is in the starting position and the distal extremity 1.3.9 of the distal elongated part 1.3.3.1 of the elongated ledge 1.3.3 of the drug container holder 1.3 is located in the receiving element 1.7.5 of the rotator 1.7. The rotator 1.7 cannot rotate and the biasing member 1.6 is in a compressed state.

In FIG. 11B, the drug has been delivered but the biasing member 1.6 is still in a compressed state. The plunger assembly 1.5 has moved axially in the proximal direction, which has pushed the drug container holder 1.3 axially in the proximal direction, as explained earlier. Besides, the rotator 1.7 has rotated and the distal extremity 1.3.9 of the distal elongated part 1.3.3.1 is now not facing the receiving element 1.7.5 anymore. As explained earlier, the receiving element 1.7.5 defines two spaces 1.7.4.3, 1.7.4.4 in the inner pocket 1.7.4, and the width W of the first space 1.7.4.3 is similar to the width W′ of the distal elongated part 1.3.3.1. In FIG. 11B, the distal extremity 1.3.9 of the distal elongated part 1.3.3.1 is aligned with the first space 1.7.4.3, meaning the distal elongated part 1.3.3.1 is free to pass through the first space 1.7.4.3 and move axially in the distal direction.

In FIG. 11C, the biasing member 1.6 is now in an extended state. The extension of the biasing member 1.6 was made possible by the ability of the distal elongated part 1.3.3.1 to pass through the first space 1.7.4.3 and move axially in the distal direction. In FIG. 11C, the distal elongated part 1.3.3.1 has fully gone through the rotator 1.7, and the flexible lock-out fingers 1.3.5 are passing through the first space 1.7.4.3. In order to be able to go through the first space 1.7.4.3, the lock-out fingers 1.3.5 had to flex, meaning their proximal extremities 1.3.5.2 had to touch each other.

In FIG. 11D, the flexible lock-out fingers 1.3.5 have fully passed the rotator 1.7, and they are back to their initial shape, meaning their proximal extremities 1.3.5.2 have spread apart. Thus, an axial movement of the drug container holder 1.3 in the proximal direction is not possible anymore. In this configuration, the needle assembly 1.4, connected to the drug container holder 1.3, is fully contained inside the housing 1.2, and cannot move proximally anymore. The injection device D is locked out.

It should be noted that the drug container holder 1.3 cannot move distally either anymore, because the protrusion 1.3.6 on the drug container holder 1.3 abuts the proximal end 1.7.p of the rotator 1.7.

Thus, an object of the invention is to use a rotating locking member, the rotator 1.7, and have it turned by the plunger assembly 1.5 at the end of the injection to allow for needle retraction and lock-out. The lock-out is enabled by the four flexible lock-out fingers 1.3.5 that bend along the barrel 2.2 of the drug container 2 during lock-out and allowed to pass the rotator 1.7 in only one direction.

Another object of the invention is to have a plunger rod 1.5.1 that is connected to the drug container holder 1.3 by flexible locking arms 1.3.8, that once assembled to the rotator 1.7 and the housing 1.2 are not allowed to flex radially outward anymore, hence mechanically locking the plunger assembly 1.5 to the housing 1.2.

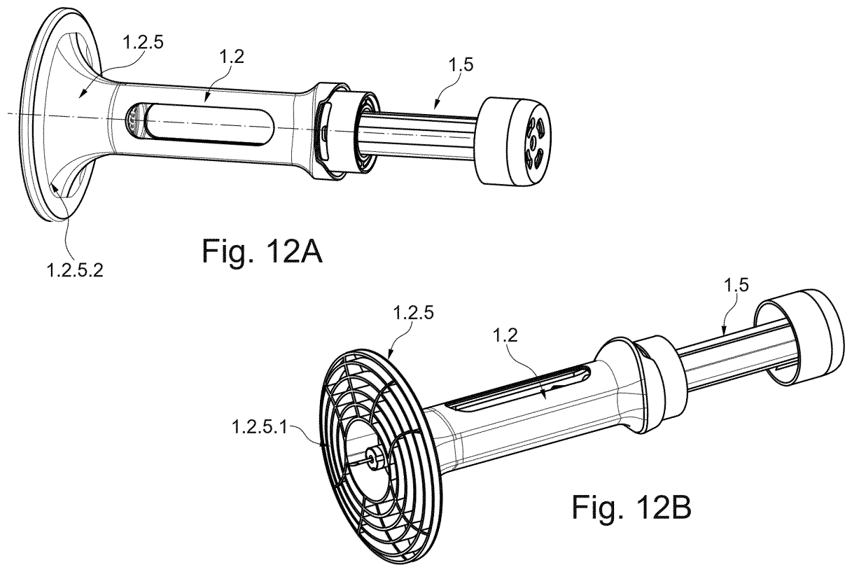

FIGS. 12A and 12B show the housing 1.2 of the injection device D according to an alternative embodiment.

This embodiment is intended for more viscous drugs where the force required for manual injections is higher, compared to the previous described embodiment. The injection device D according to this embodiment is intended to be used with both hands and has a large contact area for distribution of the applied load on the injection site.

In this embodiment, the housing 1.2 does not comprise any handle and comprises a flaring neck 1.2.5 at the proximal end. The flaring neck 1.2.5 defines, on its proximal surface, an injection site support area 1.2.5.1 to lower the pressure on the injection site during the plunger assembly 1.5 stroke. Besides, the flaring neck 1.2.5 defines, on its distal surface, a hand resting area 1.2.5.2.

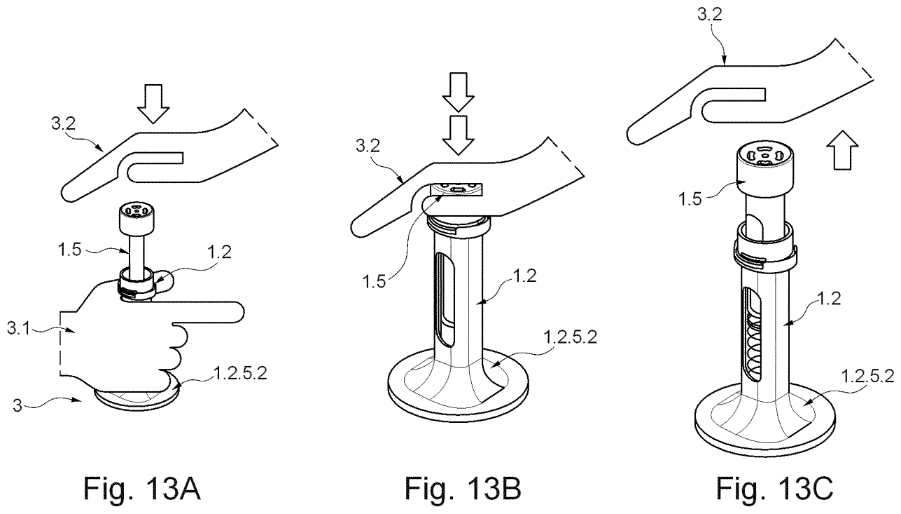

FIGS. 13A, 13B and 13C show three steps of an injection sequence intended to be performed with the embodiment of FIGS. 12A and 12B.

First, as depicted in FIG. 13A, the injection device D is supposed to be placed on an injection site, the injection site support area 1.2.5.1 contacting the injection site 3. A first hand 3.1 is supposed to hold the housing 1.2 of the injection device D and be resting on the hand resting area 1.2.5.2. Then, as depicted in FIG. 13B, a second hand 3.2 is supposed to press the plunger assembly 1.5 towards the injection site 3. Finally, as depicted in FIG. 13C, the pressure on the plunger 1.5 must be released.

The injection devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the injection devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the injection devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the injection devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fe receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-Li) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the injection devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the injection devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the injection devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the injection devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the injection devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the injection devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the injection devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3,5+2,7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

1-14. (canceled)

15. A safety device for delivering a drug, the safety device extending generally along a longitudinal axis and having a proximal end configured to point towards a dose delivery site during use of the safety device, and a distal end pointing away from the dose delivery site, the safety device comprising:
a drug container holder, generally cylindrical and extending along the longitudinal axis, and configured to receive and hold a drug container;
a ring-shaped rotator arranged around a portion of the drug container holder; and
a manually activatable plunger assembly, extending along the longitudinal axis, the plunger assembly comprising a plunger rod capable of acting on a plunger inside a barrel of the drug container for expelling a dose of the drug, and a plunger head formed on a distal end of the plunger rod and configured to be manually pushed in a proximal direction to expel the drug,
the plunger head having at least a fin extending towards the proximal end, the rotator comprising an outer pocket designed to receive the fin, and the shapes of the fin and of the outer pocket are such that a movement of the plunger head towards the rotator along the longitudinal axis triggers a rotation of the rotator when the fin contacts a corner of the outer pocket.

16. The safety device according to claim 15, wherein a proximally facing edge of the fin is angled relative to planes that are orthogonal to the longitudinal axis.

17. The safety device according to claim 15, wherein the plunger head defines an annular recess about the plunger rod and comprises a distal portion extending orthogonal to the longitudinal axis, and the fin extends in the annular recess, parallel to the longitudinal axis, from the distal portion.

18. The safety device according to claim 15, wherein the rotator has an outer surface comprising a recess forming the outer pocket, the outer pocket extending from a distal end of the rotator.

19. The safety device according to claim 15, wherein an outer surface of the rotator comprises at least a locking knob, and a housing of the safety device has a collar, the collar comprising at least an elongated through aperture forming a track for the locking knob, allowing a rotation of the rotator around the longitudinal axis but preventing an axial displacement of the rotator with regards to the housing.

20. The safety device according to claim 19, wherein a housing of the safety device comprises at least a handle, and the collar is substantially annular and extends from the handle towards the distal end, around the longitudinal axis.

21. The safety device according to claim 15, wherein an outer surface of the drug container holder has a distal elongated ledge extending parallel to the longitudinal axis, and the rotator comprises a receiving element designed to receive a distal extremity of the distal elongated part in a non-rotated position of the rotator, and a first space forming a track for the distal elongated part in a rotated position of the rotator.

22. The safety device according to claim 21, wherein an inner surface of the rotator comprises at least a recess forming an inner pocket, the receiving element protruding from said inner pocket, and the first space is defined between a first lateral edge of the inner pocket and the receiving element, the first space extending fully across the rotator along the longitudinal axis.

23. The safety device according to claim 21, wherein the drug container holder comprises two flexible lock-out fingers joining each other at a connection area corresponding to a proximal extremity of the distal elongated part, the lock-out fingers being adapted to cross the first space when the drug container holder is moved relative to the rotator only in a distal direction.

24. The safety device according to claim 23, wherein the outer surface of the drug container holder comprises a proximal elongated ledge extending parallel to the longitudinal axis, the first space forming a track for the proximal elongated part in the rotated position of the rotator after the flexible lock-out fingers have passed the rotator.

25. The safety device according to claim 24, wherein the proximal elongated part comprises a protrusion configured to prevent an axial displacement of the drug container holder in the proximal displacement once the flexible lock-out fingers have passed the rotator.

26. The safety device according to claim 15, wherein the drug container holder comprises at least two flexible locking arms extending parallel to the longitudinal axis from a distal end of the drug container holder, the locking arms being designed to surround a portion of the plunger rod, the rotator being designed to surround the locking arms and prevent the locking arms from flexing radially outward.

27. The safety device according to claim 26, wherein the locking arms have an elongated inner recess designed to complement strips extending along the plunger rod.

28. The safety device according to claim 15, further comprising a flaring neck at the proximal end, the flaring neck defining a proximally facing injection site support area, and a distally facing hand resting area.
